(19) United States
(12) Patent Application Publication
(10) Pub. No.: US 2012/0195925 A1
(43) Pub. Date: Aug. 2, 2012

(54) Vaccines with increased immunogenicity and methods for obtaining them

(76) Inventors: Artur Martynov, Kharkov (UA); Boris S. Farber, Brooklyn, NY (US); Sonya Sophya Farber, New York, NY (US)

(21) Appl. No.: 12/931,466

(22) Filed: Feb. 1, 2011

(51) Int. Cl. A61K 39/12; A61P 31/12; A61P 37/04; A61P 31/00; A61K 39/00; C12P 21/00

(52) U.S. Cl. 424/204.1; 424/184.1; 435/68.1

(57) ABSTRACT

Vaccines with increased immunogenicity, distinct in that in the capacity of a specific immunogenic component, whole vaccine antigens or vaccine antigens that have been cut into oligomer fragments are used; the mixture (assembly) of oligomer fragments or whole antigen obtained is modified by changing its molecular charge to the opposite.

### SUMMARY OF THE INVENTION

Vaccines with increased immunogenicity, distinct in that in the capacity of a specific immunogenic component, whole vaccine antigens or vaccine antigens that have been cut into oligomer fragments are used; the mixture (assembly) of oligomer fragments or whole antigen obtained is modified by changing its molecular charge to the opposite.

### TECHNICAL RESULT

An assembly of modified vaccine antigens based on dynamic self-organizing systems and the method for obtaining it, on the basis of which preventative drugs for human and animals may be obtained: vaccines with a wide spectrum of activity and improved immunogenicity and effectiveness in the prevention of pathologies such as HIV/AIDS, herpes, cytomegalovirus, and hepatitis C. Due to their ability to adapt to an organism, the application of these vaccines permits the protection of the organism even against mutant variants on infectious agents that do not yet exist in nature. This drug has a wide spectrum of activity and a low level of toxicity; it is accessible for industrial production; it is highly immunogenic and non-allergenic; it is metabolized quickly and does not contain toxic components.

### TECHNICAL FIELD

This invention is related to veterinary and human medicine—specifically, to vaccinology and pharmaceuticals—and is intended to prevent and treat infectious and other illnesses in humans and animals, in which vaccinations are applied.

### PREVIOUS LEVEL OF TECHNOLOGY

In today's world, vaccinations are one of the main methods of preventing epidemics. There are two basic groups of infectious diseases: the group of infections controlled by vaccines (whose use prevents epidemics) that belong to the required vaccination scheme, and the second group of infections, against which vaccinations are of little or no effect [¹]. Belonging to the first group of infections are conservative microorganisms and viruses whose antigen components are unchanging and a vaccine induces high levels of protective antibodies in the blood. These are infections such as diphtheria, pertussis, measles, rubella, etc. The second group of infections, against which vaccinations are ineffective, are those such as influenza, herpes infections, HIV/AIDS, and certain others [²,³,⁴]. There are many reasons for the ineffectiveness of this vaccine in preventing this group of infections. For example, the influenza virus is a polymorphic (the virus particle does not have a precise structure and form) virus with a fragmented, variable genome.

The influenza virus is very changeable and persistent (lifetime residence in the human body) [⁵]. In the bodies of humans and animals [including birds [⁶]) this virus multiplies in several stages: in the intensely productive phase, the infected cell divides the viral particles, which are capable of infecting neighboring cells [⁷]. In the persistence (latent) phase, this virus “waits around” inside the cell, meanwhile losing a part of its fragmented genome or picking up particles of human RNA in the cytoplasm [⁸]. According to statistics, a virus's antigen content changes by 5% per month [⁹]. Correspondingly, the application of standard approaches to the development of flu vaccines is not promising. Even the application of recombinant proteins and new types of gene vaccines does not prevent these drugs from aging quickly. The presence in one ampoule of several conservative proteins (for example, hemoagglutinins and neuroamidinases for the flu virus) does not permit the protection of the organism from viral aggression through inducing the production of specific antibodies. These antibodies will not have the same monoclonal specificity at all, which will be necessary at this level of virus mutation.

The change in the approach to vaccine design must be accompanied by the inclusion of antigens in the vaccine that have not yet appeared as the result of viral mutation [¹⁰]. The so-called predicative inclusion of antigens is possible through two methods: the classical, with the application of methods for the epidemiological prediction of antigenic drift, or the method of the partial modification of antigens with the obtainment of an unlimited quantity of antigen combinations in one ampoule of antigen [¹¹]. The first approach has only partially proven itself: in no case has the antigenic drift prediction coincided with the real mutational changes to flu neuroamidinases and hemoagglutinins [¹²,¹³]. If we use the technique of partial modification of the protein component of the vaccine antigen—for example, type 1 neuroamidinases—in the vaccine preparation process, then in one dose of the vaccine, in place of one protein with one antigen profile, more than a million proteins with one primary and secondary structure, but with differing replacement sites and antigen profiles will appear.

The antibodies induced by this protein will cover all possible attachment site combinations. Accordingly, the quantity of induced monoclones will be of an order higher, though the protein will remain the same. Also, any “future” epitope of the structure of a neuroamidinase will be covered by antibodies that have already been synthesized.

This approach permits us to sharply limit the quantity of antigen for vaccination, to protect the organism from viruses with a fragmented genome and highly changeable microorganisms using a small quantity of antibodies but with a significantly larger spectrum of monoclonal specificity. Roughly speaking, the vaccine will contain a selection of even those neuroamidinase antigens that do not even exist yet. Additionally, the blood of animals vaccinated earlier will already contain a necessary pool of antibodies to “future” viral strains. The use of this vaccine will allow us to successfully protect an organism from highly changeable and persistent, as well as low-immunogenic, viruses and microorganisms.

The World Health Organization has stated that research on replacing parenteral vaccines with oral ones is a priority area [¹⁴]. Non-parenteral methods of vaccination are based on the ability of antigens to penetrate through mucous membranes, mostly through the intestinal tract. The administration of oral vaccines allows the facilitation of uninterrupted antigen stimulus, which is a necessary condition for the support of the collective immune system at a high level against infections that are regulated by means of specific prevention. Moreover, this method of immunization is the simplest, and is physiologically adequate and psychologically attractive.

A method is known [¹⁵] of increasing immunogenicity through the expression of a cholera toxic antigen in plants' chimeric proteins. A method of increasing immunogenicity of nucleoproteins of the hepatitis B virus through covalent binding of amino groups of this protein with a microbial haptene is known. This conjugation allowed an increase in a vaccine's immunogenicity and the induction of an antibody titer up to the level of 1:128 (when animals were vaccinated with native protein the antibody titer did not exceed 1:32). This technology may not be used to increase the immunogenicity of other (microbial and viral) vaccines because it relies exclusively on a protein that is inducted into the cell nucleus by the hepatitis B virus. In addition, the oral use of these vaccines is impossible, since the intestinal enzymes break apart the antigens. The method of conjugation itself is also insufficiently effective, as the titers of the antibody to the modified antigen only increased by a factor of 4. The method that is most closely related to the drug being patented is a method of obtaining a water-soluble adjuvant [¹⁶]. This adjutant contains fragments of peptidoglycan and polysaccharides with acetylglucosamine and acylmuramic acid. The latter was modified by succinic or phthalic anhydride rather than by an acetyl group. The peptidoglycan was extracted from the cell walls of Nocardia microorganisms and 30-50% of the peptidoglycan was acylated. The application of this method provided the opportunity to increase the immunogenicity of the microbial antigen by 25-30% against the standard injected vaccine. The titers of specific antibodies increased to up to 1:256-1:512 two weeks after vaccination. Obtainment of pure peptidoglycan is an important procedure, as it is connected with multi-stage purification. In addition, these vaccines were completely ineffective when given orally, because they were broken down by intestinal enzymes; in connecting with the fact that the adjuvant did not have covalent bonds with the antigen, a significant difference was observed between the titer of antibodies to the adjuvant (1:400) and the titer of antibodies to the microbial antigen (1:100). The distinguishing quality of both inventions comprises, first and foremost, the use of the indicated anhydride for the modification of antigen complex that vary in chemical nature: peptidoglycan in the analogue and the protein substance in the proposed invention. Moreover, in the known invention, acylation is done by the antigen complex, which is applied in the capacity of an adjuvant; in the model being patented, the remainders of organic acids in the structure of the modified antigen themselves act as adjuvant bonds.

### DISCLOSURE OF THE INVENTION

The task of the invention is to develop vaccines with increased immunogenicity and effectiveness, including oral vaccines, and methods for obtaining them.

The task set is addressed through the development of vaccines with increased immunogenicity and methods of obtaining them, distinct in that in the capacity of a specific antigen, vaccine antigens cut into oligomer fragments (polysaccharide, protein, lipopolysaccharide) or the entire antigen (bacteria, virus, a mixture of bacterial or viral proteins) is used; the mixture (assembly) of oligomer fragments obtained is modified by changing the molecular charge to the opposite through acylation with succinic anhydride or alkylation by monochloracetic acid; also in in the capacity of a specific immunogenic component, a vaccine antigen that has had its molecular charge partially changed: this change in charge is brought about by acylation or alkylation with the formation of a mixture (assembly) of vaccine antigens with various molecular charges. We used an assembly of vaccine antigens cut into oligomer fragments, which were specific immunogenic components with changes to the opposite molecular charge. “Supramolecular assembly” and “assembly” are terms from supramolecular chemistry. The objects of supramolecular chemistry are supramolecular assemblies that self-assemble out of their complements—that is, fragments that have geometrical and chemical correspondence—similar to the self-assembly of the most complex three-dimensional structures in a live cell [¹⁷,¹⁸]

This technology may be used for the creation of other vaccines: for the prevention of infections such as influenza, hepatitis, herpes virus, mumps, measles, and HIV/AIDS; for viral infections in animals: Newcastle Disease, avian infectious bursal illness, classic swine fever, African swine fever, and any other illnesses. In connection with the partially modified structure, in the reaction modification process, a huge number of varied deviations on the vaccine antigen are formed with varying immunogenicities and structures; correspondingly, the immune system induces the synthesis of a large number of monoclones in response to these new antigen determinants. Moreover, this variety of new epitopes (hundreds of thousands or even millions) allows the predictive protection of the organism against future strains of influenza and mutated HIV/AIDS viruses that do not yet exist.

### SHORT DESCRIPTION OF DRAWINGS

FIG. 1. Dependence between a Titer of Induced Specific Antibodies and the Acylation Level of a Corpuscular Pseudomonas Antigen

FIG. 2. Dependence between a Titer of Induced Specific Antibodies and the Acylation Level of a Soluble High-Molecular-Weight Pseudomonas Antigen

### BEST INVENTION IMPLEMENTATION OPTION

### EXAMPLE 1

### Obtaining a Pseudomonas Vaccine on the Basis of the Composition of Vaccine Antigens with a Change to the Molecular Charge

Pseudomonas aeruginosa was cultivated on a solid medium (plain broth with 1% glucose added). After three days, the surface of the medium was completely covered with Pseudomonas aeruginosa. From the surface of the medium in the petri dish, a lavage was done with a 0.9% solution of sodium chloride; the suspension obtained was rinsed and centrifuged three times. After a repeat suspending of the suspension, the microorganisms were heated in a thermostat over 140 minutes at a temperature of 80° C. and then were again inoculated on a PMA medium for the purpose of inactivation control. In quantity of microbial bodies, the suspension obtained measured at 10 billion cells/ml; then 0.1 ml of the suspension was diluted 100 times with a 0.9% solution of sodium chloride, and the concentration of surface proteins was determined using the biuret method and in complexing reaction with the blue phenyl bromide Flores method [3]. In conversion to proteins, an acylation reaction was conducted with a succinic anhydride solution as indicated in Example 1 [6]. Eight samples were obtained with varying levels of acylation: 1%, 3%, 5%, 7%, 9%, 11%, 13%, and 15%. Exceeding 15% completely deprives the succinylated proteins of immunological potency; therefore, it was not considered worthwhile to obtain and use those produced with a modification level of more than 15% in future. The corpuscular antigen with a different level of acylation was used further for determination of its immunological potency. Another part of the antigen was centrifuged for 40 minutes at 3 000 revolutions per minute. The sediment was removed, and the supernatant was run through a column of Sephadex G-75. The first, heaviest fraction was collected and used further for establishing the concentration of protein and the level of chemical modification. The antigen obtained was a homogeneous fraction (one polymeric substance) and had a molecular weight of 1.5 mDa and a charge of −186000. A diluted glycoprotein antigen was obtained with these levels of acylation: 1%, 3%, 5%, 7%, 9%, 11%, 13%, and 15%.

It is well known that when gel filtration is used, the separation of proteins takes place according to the sizes of the protein globules. Gel filtration [7] was conducted in columns filled with Sephadex G-75 gel. The total volume of the column of gel was determined to be Vt. Throughout the elution, the large molecules that do not penetrate the gel granules move quickly in combination with the intergranular solution and appear in the form of a narrow layer. The volume of eluent that corresponds to the appearance of this zone was determined to be V₀ (free volume). The smaller molecules passed through the column slowly and penetrated the granules of the gel, and their exit from the column took quite a long time. In connection with the fact that the level of diffusion in the gel granules depends on the size of the molecules, the substances were removed from the column in decreasing order of their molecular mass. The molecular weight of a M of the experimental protein was established through comparison of a volume of eluted Ve with the analogous parameters of the protein markers.

To separate out the antigen, a column with a diameter of 25 mm and a length of 1000 mm was used. The G-75 and G-25 Sephadex gel was prepared ahead of time thus: to a buffer solution (0.1 M tris —HCl and 0.1 M NaCl, pH=8.0) were slowly added gel granules; this was kept in a thermostat for 72 hours at a temperature of 37 degrees C. Then the gel was deaerated (gradually and carefully mixed to an excess amount of buffer solution to remove gas bubbles) in a shaker over the course of an hour. Filtration paper was placed in the bottom of the column. A small amount of buffer solution was slowly added to the column; then a small quantity (5 g) of the suspension of swollen Selphadex G-25 gel granules was added along the wall of the container. After the formation of the gel column, no less than two volumes of reaction buffer were passed through the column. Then 100 mcl of the sample solution was extracted using a micropipette. The constant speed of the elution was set through installing a drip over the column with a buffering solution and regulating the speed of the elution using a rolling regulator. The eluent was collected in 0.5 ml test tubes and analyzed with an SF-56 spectrophotometer at a wavelength of 280 nm using method [4].

The anti-Pseudomonas serum for diagnostic purposes with a titer of specific anti-Pseudomonas antibodies (1:1000) was obtained through a standard mouse immunization scheme, according to scheme [1] of the thermally inactivate corpuscular Pseudomonas vaccine with a particle concentration of 10 billion/ml, which was administered at 3; 5 and 7 days intramuscularly at a dose of 0.2 ml. Twenty mice were used to obtain reference serum.

To immunize the animals in the experiment, acylated samples of both the corpuscular antigen (ten animals per sample, eight groups) and the acylated antigen in solution (eight samples, ten animals per sample) were taken. The first group of animals were administered 8 samples of antigen (10 animals per sample perorally at 0.2 ml each) according to the Pasteur scheme (at 1; 3 and 7 days); the second group was administered samples in accordance with the scheme developed by Prof. E. M. Babich (every other day, 0.2 ml perorally over 15 days) [1]. The level of antibodies was established through two methods: hemoagglutinin reaction and antibody fluorescing. Three animals from each group were left alive until the 15-day mark, then killed with ether and used to obtain serum, in which the level of specific antibodies was also established using the abovementioned methods. For the implementation of the abovementioned, a test system was prepared for a direct hemoagglutination reaction, which was conducted in round-bottomed 96-well immunology plates, to which was added 0.02 ml each of a 0.1% suspension of thermostatted sheep erythrocytes and 0.02 ml each of a suspension of thermally inactivated Pseudomonas cells in a concentration of 10 billion cells/ml. The level of antibodies was established in sequenced tenfold (but twofold) mouse blood serum cultures, which were added to microtiter wells in the amount of 0.02 ml. The presence of agglutinates bore witness to the creation of immune complexes. Normal human immunoglobulins (a titer of anti-Pseudomonas antibodies made up from 0 to [1:10] in accordance with AND) and a serum of the blood of unvaccinated mice (titer from 0 to 1:10) were used as controls.

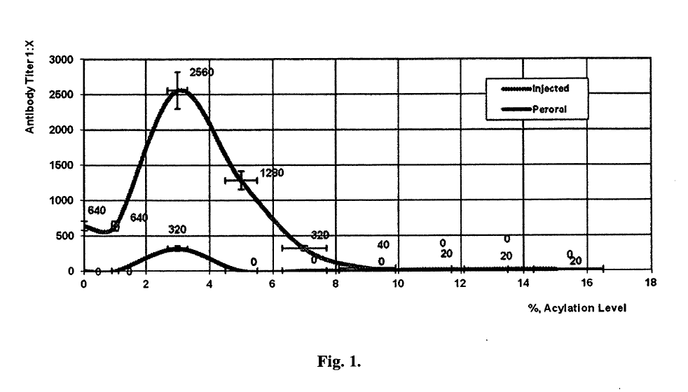

The first sample was a model of a corpuscular vaccine based on inactivated pasteurization of Pseudomonas aeruginosa. Only the surface antigens were acylated. The level of acylation varied from 1% to 15% in increments of 2%. In total, eight samples of modified antigen in solution and eight corpuscular antigens were studied. The results of the study of the immunogenic potential of the samples obtained are illustrated in the data of FIG. 1.

As may be seen in FIG. 1, 15 days after the injection of the native vaccine drug, the average titer of antibodies in the vaccinated animals came to (1:640). The oral use of the native corpuscular antigen did not cause induction of the synthesis of specific antibodies.

The chemically modified antigen from the modification stage at 1% of the concentration of protein in it induced the synthesis of the same level of antibodies as did the native antigen (1:640). Modification of surface antigens by 3% led to the induction of antibodies on the level (1:320) for the oral variation and on the level of (1:2560) for the injected variation.

For the derivative antigens with other levels of acylation, induction of the synthesis of specific anti-Pseudomonas antibodies was not observed when they were administered orally. The injection variation was effective even in derivatives with a 15% level of acylation. The derivative with an acylation level of 5% induced antibody synthesis at a level of (1:1280); the derivative with a 7% level of acylated antigen did so at a level of (1:320); the 9% derivative had a level of (1:40), and other variations (from 11-15% modified) had a level of (1:20).

Thus the most effective derivative turned out to be the one with the 3% acylation level, which induced the synthesis of specific antibodies both when the classic Pasteur vaccination scheme was used with injection and when the E. Babich oral vaccination scheme was used.

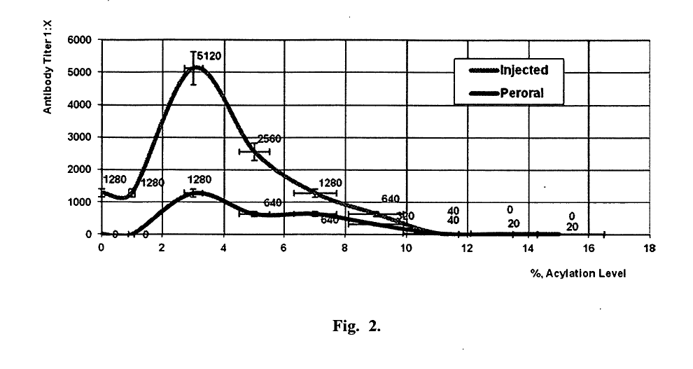

In the next figure, FIG. 2, is presented the dependence between the level of initiating specific antibodies and the level of acylation of soluble modified antigen (first fraction).

This antibody-candidate variant is a chemical monocomponent vaccine based on a modified high-molecular glycoprotein antigen with a mass of 1.5 mDa and a molecular charge of 186000. It is the higher molecular weight or the first fraction that leaves the column upon division of the fraction that has turned out to be the most immunologically potent. In the study of the structure of the antigen obtained, the correlation between the change in charge and the mass of the antigen was confirmed, which in turn confirms the condition of the cessation of the chemical reaction of antigen structural modification. An interesting fact discovered during the injection vaccination of the mice should be noted: administering the unmodified vaccine (on the seventh day, the third time) caused an allergic reaction in some of the animals in the form of tremors, a fall in motor activity, and a refusal of food over the course of one day.

The introduction of all of the modified variants of the vaccine did not cause side effects in animals. The acylated variant of the antigen in solution with a 1% level of acylation (FIG. 14), like the non-acylated antigen, caused the induction of the synthesis of an identical quantity of antibodies in titer (1:1280). The oral administration of the antigen in solution and the antigen with an acylation level of 1% did not lead to a substantial induction of the synthesis of specific anti-Pseudomonas antibodies. The derivative of the antigen in solution with an acylation level of 3% activated the synthesis of antibodies at a level of (1:5120) in the injected form and (1:1280) in oral use. The 5% acylated derivative induced the synthesis of specific antibodies at a level of (1:640) for the oral form of administration and at a level of (1:2560) for the injected form. The 7% acylated derivative induced the synthesis of specific antibodies at a level of (1:640) for the oral form of administration and at a level of (1:1280) for the injected form. The 11% acylated derivative correspondingly induced the synthesis of specific antibodies at a level of (1:320) for the oral form of administration and at a level of (1:640) for the injected form. The level of antibody synthesis was equal in vaccinated animals in the oral and injection administrations only when the vaccine drug with an acylation level of 13% was used, coming to 1:40; at the 15% acylation level, only the injected form of the vaccine candidate was immunogenic: it induced antibody synthesis at a level of (1:20).

Thus the characteristic distinction of the high-molecular antigen in solution turned out to be the induction of antibody synthesis not only by derivatives with a 3% level of acylation, but by derivatives with a 5%, 7%, 11%, and 13% acylation level in oral administration in the abovementioned vaccination scheme as described by E. M. Babich. The antibody titer of the most effective derivative with a 3% acylation level was four times more effective in injection form than it was in oral form. In comparison with the corpuscular modified antigen, the 3% acylated derivative of the antigen in solution was twice as immunogenically effective in injection form and four times more effective in oral form. Despite an antigen load that is significantly higher than in the injected form, the oral administration of partially acylated antigen in solution turned out to be effective, and induced a level of antibodies that has a perspective of protecting an organism from Pseudomonas infection in the long term (a year or more). The use of a oral vaccine is without a doubt a promising direction for the improvement of immuno-biological substances.

Among the variations of acylated antigens, the dissolved glycoprotein antigen succinylated to 3% of the mass of the protein was chosen, which with a oral administration for 15 days caused the induction of the synthesis of specific antibodies in a titer of (1:1280) and to a titer of (1:5120) in injection form.

### EXAMPLE 2

### Obtaining a Diphtheria Vaccine on the Basis of the Composition of Vaccine Antigens with a Change to the Molecular Charge

The microbial mass is obtained from a strain of RW-8 Weisen variant through culturing the C. diphtheriae bacteria on Lingood broth with the addition of 0.3% glucose or maltose. Culturing was conducted at a temperature of 37° C. over the course of 36 hours, after which the microbial mass was divided from the toxin through centrifuging (600 rpm for 30 minutes). The precipitate obtained (n-gram raw mass) had ethanol added to it (a concentration of 96°) in the volume of (2-4) n ml. It was then left in a refrigerator for 24 hours at a temperature of 4° C. and then centrifuged according to the established regime. The microbial precipitate had (2-10) n ml of physical solution added. The pH was brought to 7.2-7.4; it was cooled to a temperature of 4-6° C., left to stand for 3-4 hours, and then centrifuged (6000 rpm for 30 minutes). To this precipitate, a 0.8% solution of ethylenediaminetetraacetic-disodium salt (EDTA-Na2) is added while the precipitate is ground in a porcelain mortar until a viscous white mass is obtained. They were kept in the refrigerator at a temperature of 4°-6° C. for 18 hours. The extract is centrifuged at 6000 rpm for 30 minutes, after which dialysis is conducted with distilled water at a pH of 7.2-7.5, and it is thickened with polyethylene glycol with a molecular mass of 15000 Da or Sephadex G 200 Superfine 1 to a volume of n ml. The EDTA extract is re-precipitated at a pH of 7.0. Proteins (75.3±3.4)%, lipids (23.3±4.1)%, and carbohydrates (1.4±0.4)% make up the separated antigen complexes. A water suspension of somatic antigens is prepared, orienting toward obtainment of their necessary concentration for conducting oral vaccination (5.0 mg/l); the pH is brought to 8.5-9.0 using a 1.0% solution of sodium hydroxide or 1.0% acetic acid; the exact content of the protein substance is established by spectrophotometer (wavelength: 230 nm). The somatic complex is modified by succinic anhydride in relation to the protein it contains. The immunogenic properties of the complexes achieved are determined in chinchilla rabbits with a weight of 3.0-3.5 kg. The creation of ground-immunity in animals is conducted according to a scheme of two-time introduction of the antigen in a dosage of 5.0 mg an hour before feeding with a 24-hour interval over the course of five days. Serological studies are done on the seventh, fourteenth, and twenty-first days after the last vaccination using a standard diphtheria erythrocyte diagnostic with a titer of 1:3200 (see Table 1).

TABLE 1
Titers of Diphtheria Antibodies in the Blood Serum of Rabbits after Vaccination

| Percentage | | | | | | | |
|---|---|---|---|---|---|---|---|
| Ratio | Antigen | | | | | | |
| of Modifier | Dosage | Day of | Number | Antigens Obtained in These Titers | | | |
| to Antigen | (in mg) | Observation | of Animals | 1:10-1:40 | 1:80-1:160 | 1:320-1:640 | 1:1280-1:2560 |
| 1.0% | 5.0 | 7th | 5 | 2 | 0 | 0 | 0 |
| | | 14th | 5 | 2 | 1 | 0 | 0 |
| | | 21st | 5 | 2 | 0 | 0 | 0 |
| 2.0% | 5.0 | 7th | 5 | 0 | 0 | 1 | 4 |
| | | 14th | 5 | 0 | 0 | 3 | 2 |
| | | 21st | 5 | 0 | 1 | 4 | 0 |
| 3.0% | 5.0 | 7th | 5 | 0 | 0 | 2 | 3 |
| | | 14th | 5 | 0 | 0 | 2 | 3 |
| | | 21st | 5 | 0 | 0 | 4 | 1 |
| 4.0% | 5.0 | 7th | 5 | 0 | 1 | 1 | 3 |
| | | 14th | 5 | 0 | 1 | 1 | 3 |
| | | 21st | 5 | 0 | 1 | 2 | 2 |
| 5.0% | 5.0 | 7th | 5 | 3 | 2 | 0 | 0 |
| | | 14th | 5 | 4 | 0 | 0 | 0 |
| | | 21st | 5 | 4 | 0 | 0 | 0 |
| Unmodified | 15.0 | 7th | 10 | 0 | 0 | 0 | 0 |
| Antigen | | 14th | 10 | 0 | 0 | 0 | 0 |
| | | 21st | 10 | 0 | 0 | 0 | 0 |

The results achieved bear witness to the fact that the modified antigens have various abilities to affect the humoral immune system. The application of the modifier in the amount of 1.0% or less, as well as of 5.0% and more in relation to the mass of the protein component did not lead to the induction of antigen titers in animals, while acylation of 2.0-4.0% of the protein mass led to induction of defensive titers in the blood of the orally vaccinated rabbits to a titer of 1:2560; these were preserved up to 21 days.

### INDUSTRIAL APPLICABILITY

This invention is related to human and veterinary medicine—specifically to vaccinology—and may be used for the creation of drugs for the specific prevention of infectious, oncological, and other diseases. This method allows the modification of existing vaccine antigens directly at a pharmaceutical company on accessible equipment; it significantly decreases the cost of the vaccines obtained, lessens their side effects, and permits the creation of oral forms of the vaccine. Unique equipment is not needed for the production of this invention.

### REFERENCES

1. Robbins, J. B., R. Schneerson, and S. C. Szu. 1995. Perspective: hypothesis: serum IgG antibody is sufficient to confer protection against infectious disease by inactivating the inoculum. J. Infect. Dis. 171:1378-1398.
2. Del Val, M., H. J. Schlicht, H. Volkmer, M. Messerle, M. J. Reddehase, and U. H. Koszinowski. 1991. Protection against lethal cytomegalovirus infection by a recombinant vaccine containing a single nonameric T-cell epitope. J. Virol. 65:3641-3646
3. Larsen, D. L., A. Karasin, and C. W. Olsen. 2001. Immunization of pigs against influenza virus infection by DNA vaccine priming followed by killed-virus vaccine boosting. Vaccine 19:2842-2853
4. Cicin-Sain, L., Brune, W., Bubic, I., Jonjic, S., Koszinowski, U. H. (2003). Vaccination of Mice with Bacteria Carrying a Cloned Herpesvirus Genome Reconstituted In Vivo. J. Virol. 77: 8249-8255
5. Levin S A, Dushoff J, Plotkin J B. Evolution and persistence of influenza A and other diseases. Math Biosci. 2004 March-April; 188:17-28.
6. Terregino C, Toffan A, Beato M S, De Nardi R, Drago A, Capua I. Conventional H5N9 vaccine suppresses shedding in specific-pathogen-free birds challenged with HPAI H5N1 A/chicken/Yamaguchi/7/2004. Avian Dis. 2007 March; 51(1 Suppl):495-7.
7. Medvedeva M N, Petrov N A, Vasilenko S K, Simanovskaia V K, Golubev D B. The characteristics of the hemagglutinin from persistent variants of the influenza virus A/Victoria/35/72 (H3N2). Vopr Virusol. 1990 September-October; 35(5):374-6.
8. Aronsson F, Robertson B, Ljunggren HG, Kristensson K. Invasion and persistence of the neuroadapted influenza virus A/WSN/33 in the mouse olfactory system. Viral Immunol. 2003; 16(3):415-23.
9. Cox MM. Vaccines in development against avian influenza. Minerva Med. 2007 April; 98(2): 145-53.
10. Gronvall G K, Borio L L. Removing barriers to global pandemic influenza vaccination. Biosecur Bioterror. 2006; 4(2):168-75.
11. Martynov A. V., Babych E. M., Smelyanskaya M. V. Increase of vaccines adjuvanticity by succinylation of vaccine antigen//Rejuvenation Research.—August 2005, Vol. 8, No. 1:P.14-17 (Poster of Conference)
12. Taubenberger J K, Morens D M, Fauci A S. The next influenza pandemic: can it be predicted? JAMA. 2007 May 9; 297 (18):2025-7.
13. Vardavas R, Breban R, Blower S. Can influenza epidemics be prevented by voluntary vaccination? PLoS Comput Biol. 2007 May 4; 3(5):e85.
14 Scientific research and development in the vaccine field.// Materials of the 87th Session of the Executive Committee of the World Health Organization on 21 Nov. 1990. —11 pp.
15 U.S. Pat. No. 6,395,964, May 28, 2002 C12N 005/04; C12N 015/82; C12N 015/87; A01H 005/00. Oral immunization with transgenic plants/Arntzen; Charles J.; Mason; Hugh S.; Tariq; Haq A.; Clements; John D. The Texas A&M University System (College Station, Tex.); The Administrators of the Tulane Fund (New Orleans, La.) Appl No.: 817906, Aug. 4, 1997.
16 U.S. Pat. No. 4,094,971 Jun. 13, 1978. A61K 039/02. Immunological adjuvant agents active in aqueous solution Chedid; Louis A.; Audibert; Francoise Marguerite Agence Nationale de Valorisation de la Recherche Appl. No.: 717509 Aug. 25, 1976.
17 http://dic.academic.ru/dic.nsf/ruwiki/79240
18 Jean-Marie Lehn. Supramolecular Chemistry. Concepts and Perspectives.—Weinheim; New York; Basel; Cambridge; Tokyo: VCH Verlagsgesellschaft mbH, 1995.—P. 103 (Chapter 7)

1. Vaccines with increased immunogenicity, distinct in that in the capacity of a specific immunogenic component, vaccine antigens that have been cut into oligomer fragments; the mixture (assembly) of oligomer fragments obtained is modified by changing its charge to the opposite.

2. Vaccines with increased immunogenicity according to claim 1, distinct in that in the capacity of vaccine antigen, microbial glycoprotein is used.

3. Vaccines with increased immunogenicity according to claim 1, distinct in that in the capacity of vaccine antigen, a mixture of microbial glycoproteins is used.

4. Vaccines with increased immunogenicity according to claim 1, distinct in that in the capacity of vaccine antigen, a microbial peptide is used.

5. Vaccines with increased immunogenicity according to claim 1, distinct in that in the capacity of vaccine antigen, a mixture of microbial peptides is used.

6. Vaccines with increased immunogenicity according to claim 1, distinct in that in the capacity of vaccine antigen, a microbial polysaccharide is used.

7. Vaccines with increased immunogenicity according to claim 1, distinct in that in the capacity of vaccine antigen, a mixture of microbial polysaccharides is used.

8. Vaccines with increased immunogenicity according to claim 1, distinct in that in the capacity of vaccine antigen, a microbial lipopolysaccharide is used.

9. Vaccines with increased immunogenicity according to claim 1, distinct in that in the capacity of vaccine antigen, a mixture of microbial lipopolysaccharides is used.

10. Vaccines with increased immunogenicity according to claim 1, distinct in that in the capacity of vaccine antigen, a viral protein is used.

11. Vaccines with increased immunogenicity according to claim 1, distinct in that in the capacity of vaccine antigen, a mixture of viral proteins is used.

12. Vaccines with increased immunogenicity according to claim 1, distinct in that the vaccine antigen is cut into fragments using proteases.

13. Vaccines with increased immunogenicity according to claim 1, distinct in that the vaccine antigen is cut into fragments using synthetic proteases.

14. Vaccines with increased immunogenicity according to claim 1, distinct in that the charges of the oligomer fragments of the vaccine antigen are changed to their opposites through acylation.

15. Vaccines with increased immunogenicity according to claim 1, distinct in that the charges of the oligomer fragments of the vaccine antigen are changed to their opposites through alkylation.

16. Vaccines with increased immunogenicity according to claim 1, distinct in that the molecular charges of from 0.5% to 100% of the oligomer fragments of the vaccine antigen are changed to their opposites.

17. Vaccines with increased immunogenicity according to claim 1, distinct in that in the capacity a protease, trypsin is used.

18. Vaccines with increased immunogenicity according to claim 14, distinct in that acylation is caused by anhydrides of carboxylic and polycarboxylic acids.

19. Vaccines with increased immunogenicity according to claim 16, distinct in that acylation is caused by halides of carboxylic and polycarboxylic acids.

20. A method of obtaining vaccines with increased immunogenicity, distinct in that the vaccine antigen is cut into oligomer fragments; the mixture (assembly) of oligomer antigen fragments obtained is modified by partially changing their molecular charges to the opposite.

21. A method of obtaining a vaccine with increased immunogenicity according to claim 20, distinct in that in the capacity of a vaccine antigen a microbial glycoprotein is used.

22. A method of obtaining a vaccine with increased immunogenicity according to claim 20, distinct in that in the capacity of a vaccine antigen a mixture of microbial glycoproteins is used.

23. A method of obtaining a vaccine with increased immunogenicity according to claim 20, distinct in that in the capacity of a vaccine antigen a microbial peptide is used.

24. A method of obtaining a vaccine with increased immunogenicity according to claim 20, distinct in that in the capacity of a vaccine antigen a mixture of microbial peptides is used.

25. A method of obtaining a vaccine with increased immunogenicity according to claim 20, distinct in that in the capacity of a vaccine antigen a microbial polysaccharide is used.

26. A method of obtaining a vaccine with increased immunogenicity according to claim 20, distinct in that in the capacity of a vaccine antigen a mixture of microbial polysaccharides is used.

27. A method of obtaining a vaccine with increased immunogenicity according to claim 20, distinct in that in the capacity of a vaccine antigen a microbial lipopolysaccharide is used.

28. A method of obtaining a vaccine with increased immunogenicity according to claim 20, distinct in that in the capacity of a vaccine antigen a mixture of microbial lipopolysaccharides is used.

29. A method of obtaining a vaccine with increased immunogenicity according to claim 20, distinct in that in the capacity of vaccine antigen, a viral protein is used.

30. A method of obtaining a vaccine with increased immunogenicity according to claim 20, distinct in that in the capacity of vaccine antigen, a mixture of viral proteins is used.

31. A method of obtaining a vaccine with increased immunogenicity according to claim 20, distinct in that the vaccine antigen is cut into fragments using proteases.

32. A method of obtaining a vaccine with increased immunogenicity according to claim 20, distinct in that the vaccine antigen is cut into fragments using synthetic proteases.

33. A method of obtaining a vaccine with increased immunogenicity according to claim 20, distinct in that the charges of the oligomer fragments of the vaccine antigen are changed to their opposites through acylation.

34. A method of obtaining a vaccine with increased immunogenicity according to claim 20, distinct in that the charges of the oligomer fragments of the vaccine antigen are changed to their opposites through alkylation.

35. A method of obtaining a vaccine with increased immunogenicity according to claim 20, distinct in that the molecular charges of from 0.5% to 100% of the oligomer fragments of the vaccine antigen are changed to their opposites.

36. A method of obtaining a vaccine with increased immunogenicity according to claim 20, distinct in that in the capacity a protease, trypsin is used.

37. A method of obtaining a vaccine with increased immunogenicity according to claim 33, distinct in that acylation is caused by anhydrides of carboxylic and polycarboxylic acids.

38. A method of obtaining a vaccine with increased immunogenicity according to claim 34, distinct in that acylation is caused by halides of carboxylic and polycarboxylic acids.

39. Vaccines with increased immunogenicity distinct in that in the capacity of a specific immunogenic component, the vaccine antigen is used with a partial change of the molecular charge to the opposite charge with the formation of a mixture (assembly) of vaccine antigens with various molecular charges.

40. Vaccines with increased immunogenicity according to claim 39, distinct in that in the capacity of vaccine antigen, a microbial glycoprotein is used.

41. Vaccines with increased immunogenicity according to claim 39, distinct in that in the capacity of vaccine antigen, a mixture of microbial glycoproteins is used.

42. Vaccines with increased immunogenicity according to claim 39, distinct in that in the capacity of vaccine antigen, a microbial peptide is used.

43. Vaccines with increased immunogenicity according to claim 39, distinct in that in the capacity of vaccine antigen, a mixture of microbial peptides is used.

44. Vaccines with increased immunogenicity according to claim 39, distinct in that in the capacity of vaccine antigen, a microbial polysaccharide is used.

45. Vaccines with increased immunogenicity according to claim 39, distinct in that in the capacity of vaccine antigen, a mixture of microbial polysaccharides is used.

46. Vaccines with increased immunogenicity according to claim 39, distinct in that in the capacity of vaccine antigen, a microbial lipopolysaccharide is used.

47. Vaccines with increased immunogenicity according to claim 39, distinct in that in the capacity of vaccine antigen, a mixture of microbial lipopolysaccharides is used.

48. Vaccines with increased immunogenicity according to claim 39, distinct in that in the capacity of vaccine antigen, viral protein is used.

49. Vaccines with increased immunogenicity according to claim 39, distinct in that in the capacity of vaccine antigen, a mixture of viral proteins is used.

50. Vaccines with increased immunogenicity according to claim 39, distinct in that the charge of the vaccine antigen is changed to its opposite through acylation.

51. Vaccines with increased immunogenicity according to claim 39, distinct in that the charge of the vaccine antigen is changed to its opposite through acylation.

52. Vaccines with increased immunogenicity according to claim 39, distinct in that the molecular charges of from 0.5% to 100% of the vaccine antigen are changed to their opposites.

53. Vaccines with increased immunogenicity according to claim 50, distinct in that acylation is caused by anhydrides of carboxylic and polycarboxylic acids.

54. Vaccines with increased immunogenicity according to claim 51, distinct in that alkylation is caused by halides of carboxylic and polycarboxylic acids.

55. A method of obtaining vaccines with increased immunogenicity, distinct in that the vaccine antigen is modified through partially changing its molecular charge to the opposite with formation of a mixture (assembly) of the mixture of vaccine antigens with various molecular charges.

56. A method of obtaining a vaccine with increased immunogenicity according to claim 55, distinct in that in the capacity of a vaccine antigen a microbial glycoprotein is used.

57. A method of obtaining a vaccine with increased immunogenicity according to claim 55, distinct in that in the capacity of a vaccine antigen a mixture of microbial glycoproteins is used.

58. A method of obtaining a vaccine with increased immunogenicity according to claim 55, distinct in that in the capacity of a vaccine antigen a microbial peptide is used.

59. A method of obtaining a vaccine with increased immunogenicity according to claim 55, distinct in that in the capacity of a vaccine antigen a mixture of microbial peptides is used.

60. A method of obtaining a vaccine with increased immunogenicity according to claim 55, distinct in that in the capacity of a vaccine antigen a microbial polysaccharide is used.

61. A method of obtaining a vaccine with increased immunogenicity according to claim 55, distinct in that in the capacity of a vaccine antigen a mixture of microbial polysaccharides is used.

62. A method of obtaining a vaccine with increased immunogenicity according to claim 55, distinct in that in the capacity of a vaccine antigen a microbial lipopolysaccharide is used.

63. A method of obtaining a vaccine with increased immunogenicity according to claim 55, distinct in that in the capacity of a vaccine antigen a mixture of microbial lipopolysaccharides is used.

64. A method of obtaining a vaccine with increased immunogenicity according to claim 55, distinct in that in the capacity of a vaccine antigen a viral protein is used.

65. A method of obtaining a vaccine with increased immunogenicity according to claim 55, distinct in that in the capacity of a vaccine antigen a mixture of viral proteins is used.

66. Vaccines with increased immunogenicity according to claim 55, distinct in that the charge of the vaccine antigen is changed to its opposite through acylation.

67. Vaccines with increased immunogenicity according to claim 55, distinct in that the charge of the vaccine antigen is changed to its opposite through alkylation.

68. A method of obtaining a vaccine with increased immunogenicity according to claim 55, distinct in that the molecular charges of from 0.5% to 100% of the vaccine antigen are changed to their opposites.

69. A method of obtaining a vaccine with increased immunogenicity according to claim 66, distinct in that acylation is caused by anhydrides of carboxylic and polycarboxylic acids.

70. A method of obtaining a vaccine with increased immunogenicity according to claim 67, distinct in that acylation is caused by halides of carboxylic and polycarboxylic acids.
